(19) United States
(12) Patent Application Publication
(10) Pub. No.: US 2008/0015526 A1
(43) Pub. Date: Jan. 17, 2008

(54) SUCTION CANISTER

(76) Inventors: Solomon Reiner, Brooklyn, NY (US); Moshe Y. Richard, Brooklyn, NY (US); Baruch Y. Zuckerman, Brooklyn, NY (US)

Correspondence Address: LADAS & PARRY, 26 WEST 61ST STREET, NEW YORK, NY, 10023, US

(21) Appl. No.: 11/750,647

(22) Filed: May 18, 2007

Related U.S. Application Data
(60) Provisional application No. 60/802,841, filed on May 23, 2006.

(51) Int. Cl. A61M 1/00

(52) U.S. Cl. 604/320

(57) ABSTRACT

A suction canister is formed by a container on which is secured a lid provided with a plurality of ports, one of which is corrected to a suction source and another of which is connected by a hose used to suction fluids at a patient bedside. The port connected to the patient hose is acutely inclined at the top of the lid at an angle of 15° -30° whereas the other ports are upright. The lid is provided with tabs at its interior serving as cams to tightly fit the lid on the mouth of the container by pressing the lid on the container without twisting the lid. A shield on the inside of the lid prevents splashing suctioned liquid from prematurely contacting a hydrophobic shut off filler installed in the suction ports.

### FIELD OF THE INVENTION

The invention relates to a suction canister particularly for use in hospitals and other medical environments.

### BACKGROUND

Suction canisters are known for floor, cabinet and wall mounting, generally for medical uses to provide suction at a patient bedside for various purposes such as wound cleansing, sanitation purposes, aspiration and the like.

The canister includes a plastic or glass container which can be of different sizes onto which a plastic lid is fitted. The lid is formed with tubular fittings or ports connectable to a suction inlet hose and a patient outlet hose.

### SUMMARY OF THE INVENTION

An object of the invention is to provide a suction cannister of the above type which avoids problems associated with conventional canisters.

It is a particular object of the invention to avoid kinking of the hose which connects the lid to the patient.

Another specific object of the invention is to provide a tight fitting relation between the lid and the container to avoid separation therefrom, particularly during the use of the suction cannister.

A further object of the invention is to provide a means by which the lid can be tightly fitted onto the mouth of the container by pushing the lid onto the container without twisting the lid or the container.

In order to meet the above and further objects of the invention, there is provided a suction canister comprising a container having an open top and a lid secured on the container at the open top to provide a tight fit engagement therebetween. The lid has a plurality of ports adapted for connection with various hoses, a first said port being a patient port connectable to a hose for providing suction for patient use, and a second said port being a suction port connectable to a hose connected to a suction system providing suction pressure. A plurality of caps are attached to the lid for fluid tight connection to respective ports to selectively close the ports. The patient port extends from the lid at an acute angle relative to a surface of the lid. The suction port contains a hydrophobic shut-off filter which closes the suction port when contacted by liquid suctioned into the container.

According to a specific and preferred embodiment the patient port projects at an angle of 20° from the lid thus avoiding kinking of the patient hose.

In further accordance with the invention, the lid has an outer rim and a circular inner wall facing the rim and spaced therefrom to receive the open top of the container mouth therebetween, and a plurality of tabs are distributed around the lid, the tabs having inclined cam surfaces extending from the rim towards the inner wall at an angle to press the mouth of the container against the wall to provide the tight fit of the lid on the container.

In further accordance with the invention, the cam surfaces are inclined towards the inner wall in a direction of insertion of the container into the lid whereby the lid is tightly fitted on the container by pressing the lid onto the container without twisting the lid.

The invention further provides that the container and lid are made of inexpensive material so that the canister is adapted for one-time use only.

### BRIEF DESCRIPTION OF THE FIGURES OF THE DRAWING

FIG. 1 is a top front perspective view of a canister according to the invention.

FIG. 2 is a top plan view of the lid.

FIG. 3 is a side elevational view of the lid as seen from the bottom of FIG. 2.

FIG. 4 is a side elevational view of the lid as seen from the right in FIG. 2.

FIG. 5 is a sectional view taken on line A-A in FIG. 2.

FIG. 6 is a sectional view taken on line B-B in FIG. 2.

FIG. 7 is a bottom plan view of the lid.

### DETAILED DESCRIPTION

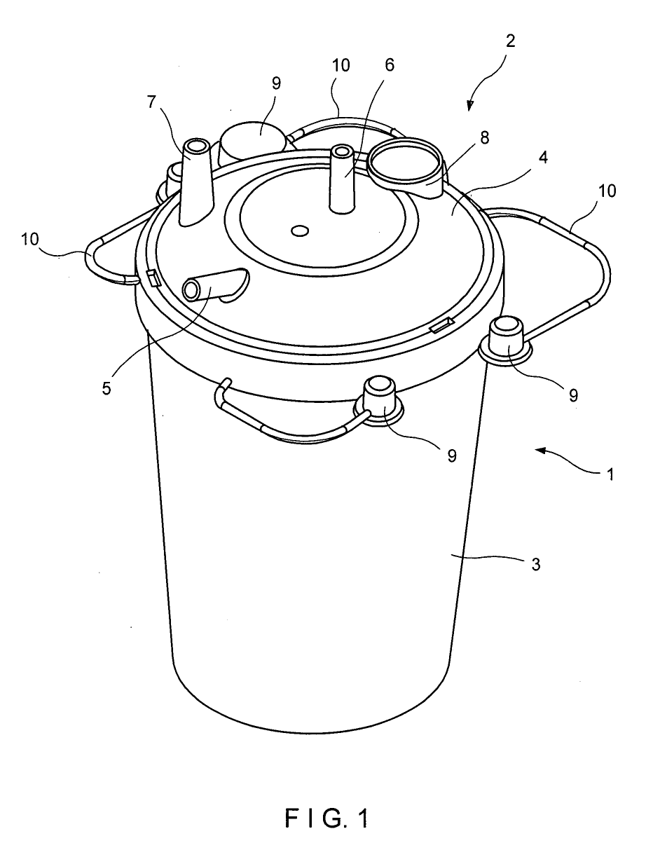

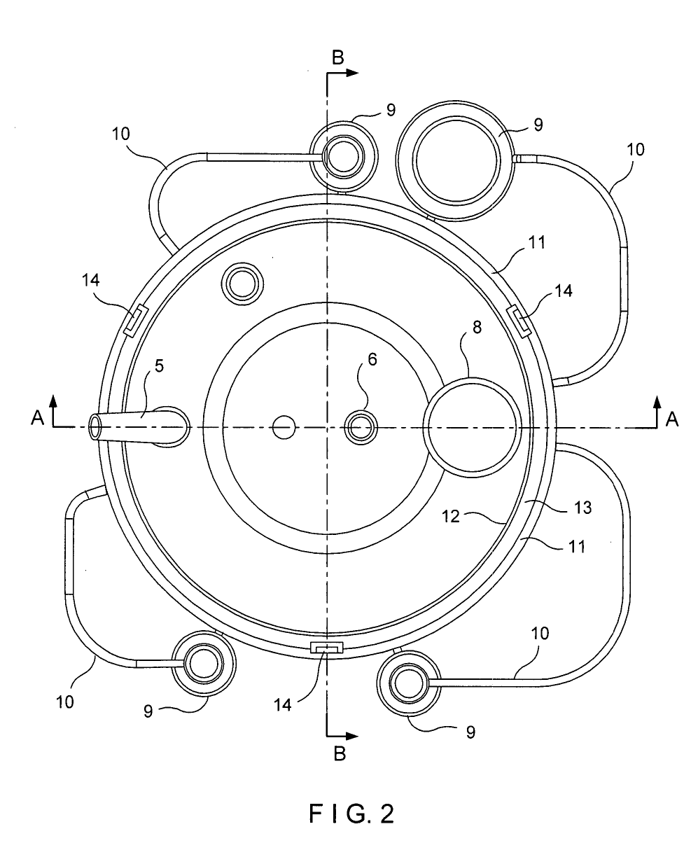

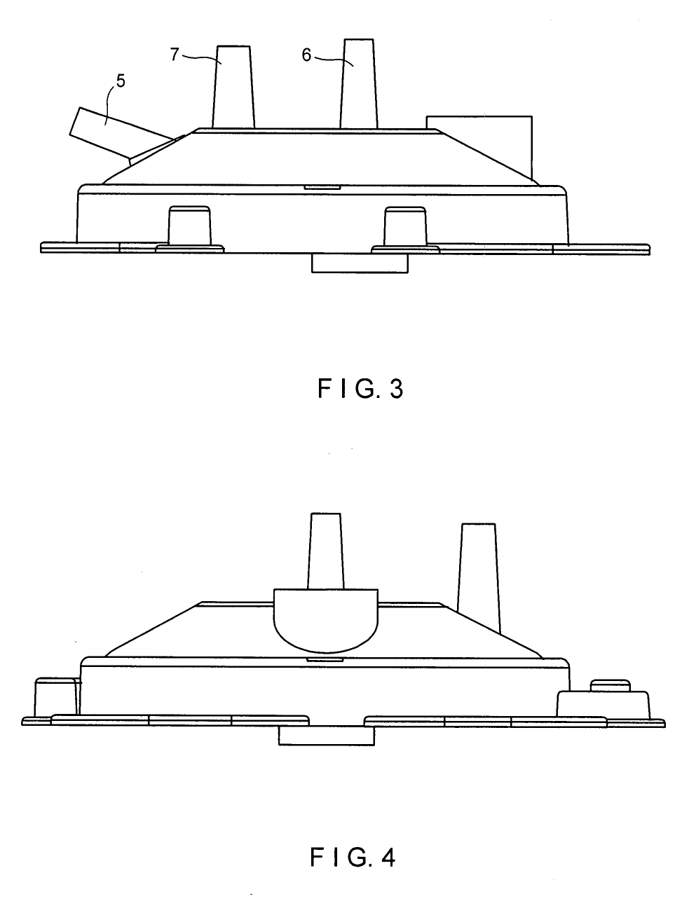

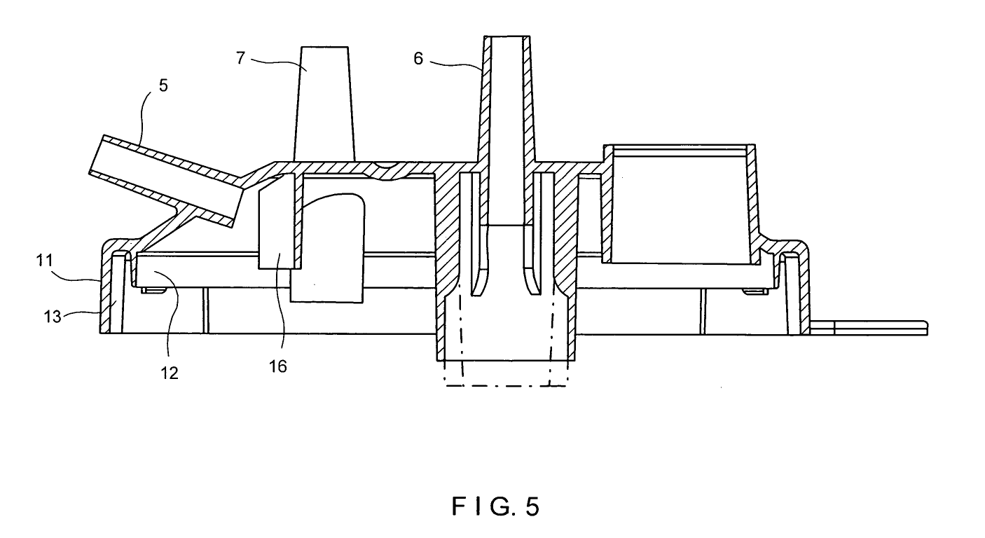

FIG. 1 illustrates an assembled canister 1 in which a lid 2 is installed on a container 3. The lid and container are made of inexpensive plastic material, such as, styrene so that the canister can be used one time and then disposed of. The container can be made in different sizes and shape depending on the particular use of the canister. Preferably, the container 3 is transparent.

Reference will next be made more specifically to the construction of the lid 2.

The lid 2 is of generally circular configuration with a domed top 4. The lid 2 is formed with a number of ports which project as hollow tubular fittings at the top of the lid which communicate with the interior of the lid and the interior of container 3. A first port 5 is a patient port to which can be connected a hose (not shown) for transporting suction to a patient. A second port 6 is a suction port which can be connected by another hose (not shown) to a suction mechanism (not shown) to develop suction in the container to suction fluids via port 5 into the container. A third port 7 is an auxiliary patient port and a large diameter port 8 serves as a discharge port for dispensing liquid which has filled the container 3. A plurality of caps 9 are connected by integral plastic connector cords or lines 10 to enable the caps 9 to be tightly fitted on respective associated ports for a purpose to be explained later.

As seen in the drawings, the ports 6, 7 extend upwardly from the lid, generally parallel to the axis of the lid and perpendicular to the edge of the lid where the lid is attached to the mouth of the container. Instead of being upright, the patient port 5 is inclined with respect to the lid. In this regard it has been determined that in order to prevent kinking of the patient hose to the patient site and allowing the patient hose to extend substantially in a straight line from the canister to the patient site, the angle of the patient port 5 extends from the top of the lid at angle between 15° and 30° and preferably 20°. This angle is that measured between the axis of the patient port 5 and a perpendicular to the axis of the lid which corresponds to the edge of the lid where the lid is mounted on the mouth of the container. If the angle of the patient port 5 is less than 15°, attachment of the patient hose to port 5 becomes difficult and if the angle exceeds 30°, kinking of the patient hose becomes problematic.

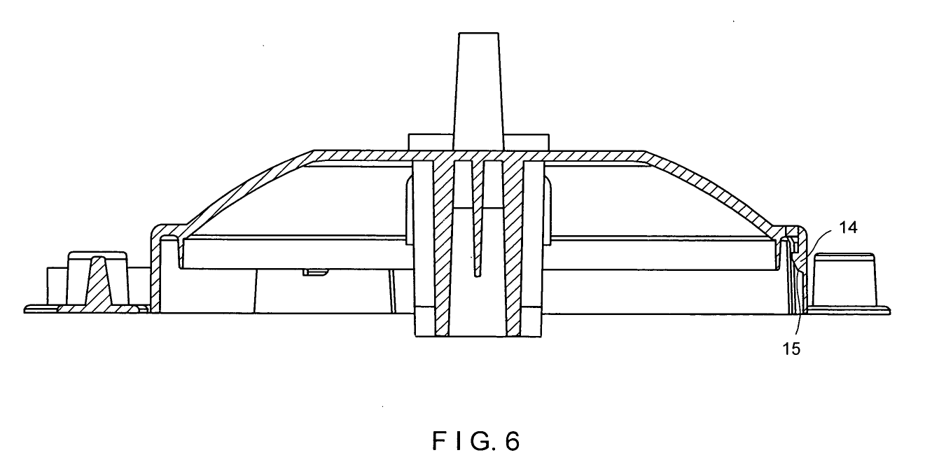

The lid 2 is formed with an outer circular rim 11 and with an inner circular wall 12 facing rim 11 and forming a space 13 therebetween. The space 13 is sized to receive the mouth of the container therein. In order to fit the lid on the mouth of the container, the mouth of the container is inserted into space 13 and in order to tightly secure the lid 2 on the container 3, a plurality of tabs 14 are distributed around the rim of the container. In the particular embodiment, three tabs 14 are equally distributed at 120° spacing on the rim. A greater number of tabs can be provided if desired. Each tab 14 is formed with an inclined cam surface 15 (see FIG. 6) which is tapered to widen in the direction of insertion of the container mouth into the space 13. Hence, when the lid is installed on the container mouth, the lid will be tightly secured to the container. The installation is achieved by simply pressing the lid onto the container without relative twisting between the lid and the container.

When the lid is secured on the container, the suction hose can be connected to the suction inlet port 6 and the patient hose can be connected to the patient port 5. The suction canister is now ready for operation and the suction hose can be used for various medical purposes to suction liquid into the canister. The suction inlet 6 contains a conventional hydrophobic shut-off filter which serves to filter the suction air and when the container is filled with liquid suctioned at the patient port 5 to a level of the hydrophobic shut-off filter, the filter softens and seals off the suction inlet from the interior of the canister to block suction at the suction inlet from connection into the canister. At such time, the patient and suction hoses are disconnected from their respective ports and the caps 9 are secured on the ports to close the ports. The cap on the discharge port 8 is then opened and the contents of the container are suitably poured into a collection reservoir or simply flushed away. The cap is then placed on the discharge port 8 and the canister is now ready for disposal.

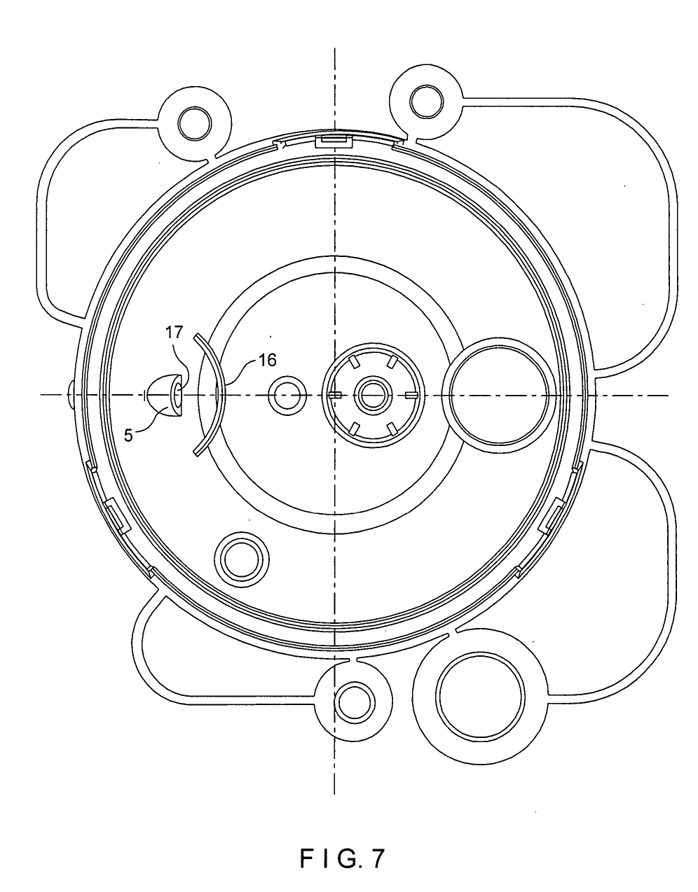

In order to prevent suctioned liquid from contacting the hydrophobic shutoff filter before the container is filled with liquid, a curved shield 16 (FIG. 7) is formed at the underside of the lid and faces the outlet 17 of the patient port 5. The shield 16 extends over an arc of nearly 180° and extends well below the outlet 17 of the patient port 5.

What is claimed is:

1. A suction canister comprising a container having an open top and a lid secured on the container at the open top, said lid being secured on the container to provide a tight fit engagement therebetween, said lid having a plurality of ports adapted for connection with various hoses, a first said port being a patient port connectable to a hose for providing suction for patient use, and a second said port being a suction port connectable to a hose connected to a suction system providing suction, and a plurality caps attached to the lid for sealing connection to respective ones of said ports, said patient port extending from the cap at an acute angle relative to the lid, said suction port containing a hydrophobic shut-off filter which closes the suction port when contacted by liquid suctioned into the container and reaching the level of the shut-off filter.

2. The suction canister of claim 1, wherein said patient port projects at an angle of about 15 to 30° from said lid.

3. The suction canister of claim 2, wherein said angle is 20°.

4. The suction canister of claim 1, comprising a curved shield on an underside of the lid facing an opening of said patient port to block contact of suctioned fluid with the hydrophobic shut-off filter.

5. The suction canister of claim 1, wherein said lid has an outer rim and a circular inner wall facing said rim and spaced therefrom to receive the open top of the container therebetween, and a plurality of tabs distributed around said lid, said tabs having inclined cam surfaces extending from said rim towards said inner wall at an angle to press the mouth of the container against said wall to provide said tight-fit of the lid on the container.

6. The suction canister of claim 5, wherein said cam surfaces are inclined towards said inner wall in a direction of insertion of the container into the lid whereby the lid is tightly fitted on the container by insertion of the mouth of the container into the lid without relative twisting of the lid and the container.

7. The suction canister of claim 1, wherein said lid has a third said port of larger diameter than the other ports for emptying liquid which has been suctioned into the canister.

8. The suction canister of claim 1, wherein said container and lid are made of inexpensive material so that the canister is adapted for one-time use.

9. The suction canister of claim 8, wherein the material of the lid and container is plastic.

10. The suction canister of claim 9, wherein the plastic material is styrene.

11. The suction canister of claim 1, wherein the lid has a further said patient port.
